(19) United States
(12) Patent Application Publication
(10) Pub. No.: US 2012/0310030 A1
(43) Pub. Date: Dec. 6, 2012

(54) Device And Method For Line Control Of An Energy Beam

(76) Inventors: Jean-Marc Fontbonne, Caen (FR); Jérôme Perronnel, Herouville-Saint-Clair (FR); Bruno Marchand, Mamaroneck, NY (US); Caterina Brusasco, Bossiere (BE)

(21) Appl. No.: 13/499,634

(22) PCT Filed: Sep. 30, 2010
(86) PCT No.: PCT/EP2010/064601

§ 371 Date: Aug. 10, 2012

(30) Foreign Application Priority Data
Oct. 1, 2009 (EP) 09172010.2

(51) Int. Cl. A61N 5/10; G01T 1/16

(52) U.S. Cl. 600/1; 250/336.1

(57) ABSTRACT

The invention relates to the field of line control of a beam, and especially to a device comprising a plurality of ionisation chambers, enabling the measurement of the dose deposited by an ionising beam and the field of said beam. At least one ionisation chamber is formed from support films having a thickness less than or equal to 100 nm.

### TECHNICAL FIELD

The present invention relates to the field of online beam monitoring. More particularly, the present invention concerns a device comprising several ionisation chambers allowing the measurement of the dose deposited by an ionising beam and the field of this beam.

### TECHNOLOGICAL BACKGROUND

Hadron-therapy is a branch of radiotherapy allowing the delivery with precision of a dose onto a target volume, a tumour, whilst preserving the surrounding healthy tissues. Hadron-therapy apparatus comprises an accelerator producing a beam of charged particles, means for transporting the beam and a radiation unit. The radiation unit delivers a dose distribution to the target volume and generally comprises means for monitoring the delivered dose. Two major modes for delivering beams of particles are used in hadron-therapy: one first delivery mode comprises so-called passive beam scattering techniques and a second more elaborate treatment mode comprises dynamic beam scanning techniques.

The passive scattering methods have recourse to an energy degrader adjusting the pathway of the particles as far as a maximum depth point of the region to be irradiated. The energy degrader is also used in combination with a range shifter wheel, a compensator and a patient-specific collimator allowing a dose distribution to be obtained which best coincides with the target volume. One major defect of this technique is that the neighbouring healthy tissues located upstream and outside the target volume may also be subjected to high beam doses. In addition, the need to use a compensator and a collimator specific to the patient's tumour and to the angle of irradiation makes this procedure complicated and costly.

One mode for delivering a dynamic beam comprises the so-called “PBS” methods (Pencil Beam Scanning) in which a narrow beam of particles oriented along axis z is scanned over a plane orthogonal to this axis z over the target volume by means of scanning magnets. By causing the energy of the beam of particles to vary, different layers in the target volume can be successively irradiated. In this manner the radiation dose can be delivered over the entirety of the target volume.

One first method of the so-called Pencil Beam Scanning technique is a method called spot scanning. With this method, the irradiation of layers of target volume is obtained by delivering a prescribed beam dose to discrete positions of this volume and interrupting the beam between each change of position.

Another Pencil Beam Scanning method is the so-called continuous scanning technique in which the beam is scanned continuously following a predefined pattern. During the scanning of a layer, the intensity of the beam may vary at every instant so as to deliver a precise dose at the right place in the target volume, such as specified in the treatment plan. In other more advanced beam delivery techniques, the scanning rate can be adjusted instant by instant, so as to have an additional degree of freedom to modulate the intensity of the beam.

With the PBS technique not only homogeneous distribution doses but also non-homogeneous doses can be delivered to a target volume. Typically, a combination of several treatments with beams from different directions is necessary to produce a “tailored” radiation dose which maximizes the dose in the target volume whilst protecting neighbouring healthy tissues. Although a three-dimensional dose distribution in the target volume resulting from radiation in a single direction may not be uniform, provision is made so that the contribution of each radiation in several directions produces a uniform dose in the target volume. A treatment which delivers beams depositing non-homogeneous doses in which integration of each beam contribution allows a homogeneous dose to be obtained in a target volume is called Intensity Modulated Particle Therapy (IMPT). The specification of the treatment is prepared by advanced treatment planning systems using optimisation algorithms to specify the number and the directions of beam treatments and the particle intensities to be delivered to each point in each layer to be irradiated.

Another example of a dynamic technique is a radiation technique which differs from PBS and is called a uniform scanning technique in which a uniform dose is delivered to a target volume layer by layer, and in which the beam is continuously scanned assuming the form of a geometric pattern. The beam does not assume the shape of the contour of the target volume but is scanned over a predefined geometric surface area and lateral conformity is obtained by means of a collimator comprising several plates or by means of a patient-specific aperture.

Through the complexity of these different techniques, the verification of the dose sent to the patient is a crucial point. The calibration of hadron-therapy apparatus is standardized and is made using a water phantom which chiefly comprises a detector, generally an ionisation chamber or an array of pixels, which may or may not be able to be moved in a large container filled with water, the density and stopping power of water being similar to those of human tissues. This calibration is performed before treatment and the treatment plan is prepared on the basis of this calibration.

Ionisation chambers are standard dosimetry detectors generally used in radiotherapy. An ionisation chamber comprises a polarisation electrode separated from a collecting electrode by a gap comprising a fluid of any type.

There are several types of ionisation chambers such as so-called cylindrical ionisation chambers and ionisation chambers comprising parallel plates. Cylindrical ionisation chambers comprise a central or axial electrode generally in the form of a very thin cylinder insulated from a second electrode of hollow cylindrical shape or cap-shaped surrounding the said central or axial electrode. Ionisation chambers comprising parallel plates have a first plate supporting a polarisation electrode, this first plate being separated from a second plate comprising one or more collecting electrodes located opposite the polarisation electrode. The plates are separated by a gap comprising a fluid of any type. The perimeter of each collecting or polarisation electrode deposited on the plates is surrounded by an insulating resin itself surrounded by a guard electrode.

The fluid contained in the gap separating the collecting and polarisation electrodes of an ionisation chamber used in dosimetry is most often a gas. When an ionising beam passes through the ionisation chamber, the gas contained between the electrodes is ionised and ion-electron pairs are formed. An electric field is generated by applying a potential difference between the two electrodes of the ionisation chamber. The presence of an electric field allows these ion-electron pairs to be separated causing them to drift onto the respective electrodes, thereby inducing a current at these electrodes which will be detected and measured.

During treatment, it is also essential to monitor the dose delivered to the patient ensuring that it corresponds to the dose prescribed in the treatment plan, for example by means of an ionisation chamber. It must also be possible to detect any deviation of the beam. The document: “A pixel chamber to monitor the beam performances in hadron therapy”, R. Bonin et al., Nucl. Instr. & Methods in Phys. Reas. A 519 (2004) 674-686, describes an ionisation chamber comprising a cathode 25 μm thick composed of a mylar film on which aluminium has been deposited, and an anode composed of a Vetronite film of thickness 100 μm sandwiched between two films of copper each 35 μm thick. Using the PCB technique, the said anode is segmented into 32×32 pixels on one side and each pixel is connected by a via passing through the Vetronite film to a conductive trace located on the other side of the anode. Each trace connects a pixel to a signal measuring device. However, this pixel ionisation chamber has some shortcomings of which the first is mechanical instability. The distance between the two electrodes is defined by an external armature. Mechanical deformation or a microphonic effect may affect the distance between the two electrodes significantly, thereby affecting the accuracy and precision of measurement. Another problem with this device is its lack of <<transparency>> with respect to a beam. The non-negligible thickness of copper present on the anode induces beam scattering.

Document WO 2006126084 partly solves these problems by replacing the copper layers forming each pixel by graphite layers. Also an intermediate layer pierced with holes surrounding each pixel is provided between the anode and the cathode thereby forming a plurality of chambers. Attachment points fix the intermediate layer to the anode and cathode so as to allow air to pass and to stabilize the distance between the anode and the cathode.

Nonetheless, this type of detector always induces angular and longitudinal beam scattering, hence the need for the possible providing of a detector the most <<transparent>> possible, in other words whose water equivalent thickness (WET) is minimal so as not to degrade the properties of the beam.

In general, the water equivalent thickness of a portion of material m of thickness lₘ through which there passes a given beam of particles of given energy is defined as the water thickness producing the same loss of energy of the beam as the portion of material m of thickness lₘ. The water equivalent thickness of a material m of portion lₘ through which an energy beam is passed is given by the following equation:

$${WET}_{m} = {l}_{m} \begin{matrix}{ρ}_{m} & {( \frac{1}{ρ} \frac{d E}{d x} )}_{m} \\ {ρ}_{w} & {( \frac{1}{ρ} \frac{d E}{d x} )}_{water}\end{matrix} \qquad ( Equation 1 )$$

Where:

pₘ is the density of the material m, in g/cm³;

p_{w} is the density of water, in g/cm³;

lₘ is the thickness of the material, in cm;

$${( \frac{1}{ρ} \frac{d E}{d x} )}_{m}$$

is the stopping power of the material on the beam relative to the density of the material m, in MeV*cm2/g;

$${( \frac{1}{ρ} \frac{d E}{d x} )}_{water}$$

is the stopping power of water on the beam relative to the density of water, in MeV*cm2/g.

Minimisation of the water equivalent thickness for an ionisation chamber can be obtained by reducing the thickness of the plates supporting the electrodes and using materials for these plates of relatively low mean atomic weight. However, there is a limit thickness for these electrode-supporting plates below which several problems may arise.

One first problem to which consideration must be given is the increase in capacitance at the electrodes on the support film. Charge differences that are too high between the two sides of one same film may lead to breakdown of the film. For a planar capacitor, capacitance is given by

$$C = {ɛ}_{0} \cdot {ɛ}_{r} \cdot \frac{A}{d}$$

where:

εO: vacuum permittivity;

εr: relative permittivity of the material;

A: area of the plate of the electrode;

d: thickness of the plate of the electrode.

A second problem is the presence of microphonic noise affecting the distance between the electrodes and reducing the precision and exactitude of measurement.

Additionally, with a support plate of reduced thickness it becomes difficult for a via to be passed through the plate to connect one or more collecting or polarising surfaces with one or more conductive traces without affecting the mechanical stability of the plate.

Document U.S. Pat. No. 6,011,265 describes a detector comprising a single ionisation chamber comprising a plurality of support films arranged in parallel and separated from each other by a gap. The described ionisation chamber comprises:
a first support film comprising an electrode DE;
a second support film comprising a collecting electrode CE composed of a plurality of elementary anodes;
one or two support films 10 contained between the said first and second support films, the said support films 10 being made in an insulating material and metallised on their two sides so as to form a first metal cladding 11 and a second metal cladding 12, the said metal clad films 10 comprising a plurality of perforated holes, the whole forming an electron multiplier;
first polarisation means B1 for polarising the electrode D2 located on the first film;
second polarisation means B2 adapted to set up an electric polarisation voltage between the said first metal cladding 11 and the said second metal cladding 12 so as to form, at each hole, an electric field condensation region in which a condensed electric field is generated, the said condensed electric field functioning so as to generate an electron avalanche from said photoelectron, considered to be a primary electron;
third polarisation means B3 adapted to create an electric polarising voltage which is applied to the said collecting electrode CE to allow the detection of the said electron avalanche.

The detector described in U.S. Pat. No. 6,011,265 may also comprise a second assembly of elementary anodes arranged on the second side of the second support film so as to form a two-dimensional detector. However, in hadron therapy techniques which notably use beam currents of high intensity, the beam monitoring devices used are ionisation chambers operating at saturation for maximum efficacy of charge collection. Therefore, phenomena of charge recombination must be minimized subsequent to ionisation of the gas present inside an ionisation chamber, which may be detrimental to saturation of the chamber and hence to precision of measurement. As a result, it is not possible for this type of beam to use an ionisation chamber in which there is amplification of the charges produced subsequent to ionisation of the gas, such as described in document U.S. Pat. No. 6,011,265.

### Aims of the Invention

It is therefore necessary to be able to produce a detector that is sufficiently transparent to a radiotherapy beam so that the dose is delivered to the patient with accuracy and precision, minimising the phenomena of scattering and deterioration of the beam. The construction of a said detector must also take into account problems of capacity, microphonic effect and mechanical stability.

It is one of the objectives of the present invention to obtain a dosimetry device comprising an assembly of ionisation chambers which enables monitoring of the dose of a beam directed onto a patient, the device not having the disadvantages of the prior art devices.

More specifically, the objective of the present invention is to minimise the water equivalent thickness of a dosimetry device so as to deliver a dose to a patient which is the most accurate and precise as possible.

An additional objective of the present invention is to obtain good detection dynamics, in particular by eliminating or reducing the intrinsic capacitance of the support plates of the ionisation chambers whilst reducing the thickness of these support plates.

A further objective of the present invention is to provide a device whose collecting electrodes maintain uniform response over their entire surface by preventing the deformation of these support plates of narrow thickness subjected to a strong electric field.

A further objective of the present invention is to provide a device able to measure with precision both the dose deposited by a beam and the field of this same beam.

A further objective of the present invention is to provide a <<universal>> device allowing measurement of the properties of a beam obtained using both a passive delivery technique and a dynamic technique.

### SUMMARY OF THE INVENTION

According to a first aspect, the present invention relates to a device for the online monitoring of an ionising beam generated by a radiation source and delivered to a target, the said device comprising a plurality of support films arranged in parallel and separated from each other by a gap; the said support films being positioned perpendicularly relative to the central axis of the ionising beam and forming a succession of ionising chambers of which at least one ionising chamber is formed using support films having a thickness of 100 μm or less; each of the support films having on its two surfaces one or more electrodes set at a potential such that the two sides of each of the support films has the same polarity; the support films being arranged so that the successive support films have alternate polarisation; the said device further having additional means capable of equilibrating the electrostatic forces present inside the said ionisation chamber formed using support films having a thickness equal to or less than 100 μm.

Preferably, in the device of the invention, the at least one ionisation chamber is made using support films having a thickness of less than 20 μm, preferably equal to or less than 15 μm, more preferably equal to or less than 10 μm, further preferably equal to or less than 5 μm, still further preferably equal to or less than 1 μm.

Preferably in the device of the invention, the additional means comprise a rigid plate, parallel to and positioned facing the support film comprising a collecting electrode on each of its sides, and taking part in the formation of the ionisation chamber made using support films having a thickness equal to or less than 100 μm; the rigid plate further comprising at least one electrode placed at a potential capable of equilibrating the electrostatic forces present inside the ionisation chamber.

Preferably, in the device of the invention, the additional means comprise a rigid or flexible plate, preferably flexible, parallel to and positioned opposite the support film comprising a polarising electrode on each of its sides, and taking part in the formation of the ionisation chamber prepared using support films having a thickness equal to or less than 100 μm; the rigid or flexible plate further comprising at least one electrode placed at a potential capable of equilibrating the electrostatic forces present inside the ionisation chamber.

Preferably, in the device of the invention, the gaps between each support film are constant.

Preferably, in the device of the invention, at least one of the support films having a thickness equal to or less than 100 μm comprises an electrode at least on one of its surfaces, preferably a collecting electrode, connected to measuring electronics via a conductive trace located on the same side of the support film as the side comprising the said electrode, so that the mechanical stability of the said support film is not detrimentally affected.

Preferably, the device of the invention comprises support films having collecting electrodes on their two surfaces alternating with support films having polarising electrodes on their two surfaces.

Preferably, in the device of the invention, each collecting electrode electrode is connected to measurement electronics by a conductive trace located on the same side of the support film as the side comprising the said collecting electrode.

Preferably, in the device of the invention, some collecting electrodes assume the shape of strips arranged in parallel.

According to another aspect, the invention concerns a device intended to measure ionising beams, the device comprising a support film having two surfaces and having a thickness equal to or less than 100 μm, preferably less than 20 μm, more preferably equal to or less than 15 μm, further preferably equal to or less than 10 μm, still further preferably equal to or less than 5 μm, still further preferably equal to or less than 1 μm; the support film comprising an electrode on at least one of its surfaces, preferably a collecting electrode, connected to measurement electronics by a conductive trace located on the same side of the support film as the side comprising the electrode.

Preferably, in the device of the invention, the electrode assumes the shape of a disc whose perimeter is separated by a gap or insulating resin from a guard layer which extends over the remainder of the support film, and the disc-shaped electrode is connected to measurement electronics by a trace located on the same side of the said support film as the side comprising the disc-shaped electrode, the trace being coated with an insulating resin, and the insulating resin is coated with a thin layer of conductive material which extends over the guard layer.

According to another aspect, the invention concerns a method for the online monitoring of an ionising beam generated by a radiation source and delivered onto a target, the method comprising the steps of:

a) providing a plurality of support films arranged in parallel and separated from each other by a gap; the support films being positioned perpendicularly relative to the central axis of the ionising beam and forming a succession of ionisation chambers of which at least one ionisation chamber is formed using support films having a thickness equal to or less than 100 μm; each of the support films having one or more electrodes on its two surfaces;

b) placing each of the support films at a potential such that the two surfaces of each of the support films has the same polarity;

c) arranging the support films such that the successive support films have alternating polarisation;

d) determining the electrostatic forces present inside the ionisation chamber formed by support films having a thickness equal to or less than 100 μm;

e) equilibrating the electrostatic forces by means of additional means.

Preferably, in the method of the invention, the at least one ionisation chamber is made using support films having a thickness less than 20 μm, preferably equal to or less than 15 μm, more preferably equal to or less than 10 μm, further preferably equal to or less than 5 μm, still further preferably equal to or less than 1 μm.

Preferably, in the method of the invention, at least one of the support films having a thickness equal to or less than 100 μm comprises an electrode on at least one of its surfaces, preferably a collecting electrode, connected to measurement electronics by a trace located on the same side of the support film as the side comprising the said electrode, so that the mechanical stability of the said support film is not detrimentally affected.

Preferably, in the method of the invention, the additional means comprise a rigid or flexible plate comprising at least one electrode placed at a potential capable of equilibrating the electrostatic forces present inside the said ionisation chamber.

Preferably, in the method of the invention, the equilibration step further comprises the application of a suitable voltage to the support films.

According to another aspect, the invention concerns the use of the device as described above for online monitoring of beams of particles obtained using passive delivery techniques.

According to another aspect, the invention concerns the use of the device as described above for online monitoring of beams of particles obtained using dynamic delivery techniques.

### BRIEF DESCRIPTION OF THE FIGURES

The following drawings are given for illustration purposes and are not in any way to be construed as limiting the scope of the present invention. Also, the proportions of the different figures are not drawn to scale.

FIG. 1 illustrates a first embodiment of the invention comprising one or two integral ionisation chambers depending on whether or not one of the support films located at the end is flexible or rigid.

FIG. 2 illustrates one surface of a support film comprising a collecting electrode connected to measurement electronics.

FIG. 3 illustrates one surface of a support film comprising a collecting electrode that is disc-shaped connected to measurement electronics.

FIG. 4 illustrates a second embodiment of the invention in which all the support films are flexible.

FIG. 5 illustrates a third embodiment of the invention comprising two integral ionisation chambers and two ionisation chambers in strip form.

FIG. 6 illustrates a fourth embodiment of the invention comprising two pairs of integral ionisation chambers and two pairs of strip ionisation chambers.

FIG. 7 illustrates a fifth embodiment of the invention comprising integral ionisation chambers, strip ionisation chambers and two reference ionisation chambers.

FIG. 8 illustrates a sixth embodiment of the invention comprising integral ionisation chambers, strip ionisation chambers, reference ionisation chambers and ionisation chambers comprising disc-shaped collecting electrodes.

FIG. 9 illustrates a seventh embodiment comprising two reference ionisation chambers surrounded by two assemblies of ionisation located on each side of these reference ionisation chambers, a first assembly of ionisation chambers comprising strip ionisation chambers and integral ionisation chambers, a second assembly comprising strip ionisation chambers and ionisation chambers comprising disc-shaped collecting electrodes.

### DETAILED DESCRIPTION OF THE INVENTION

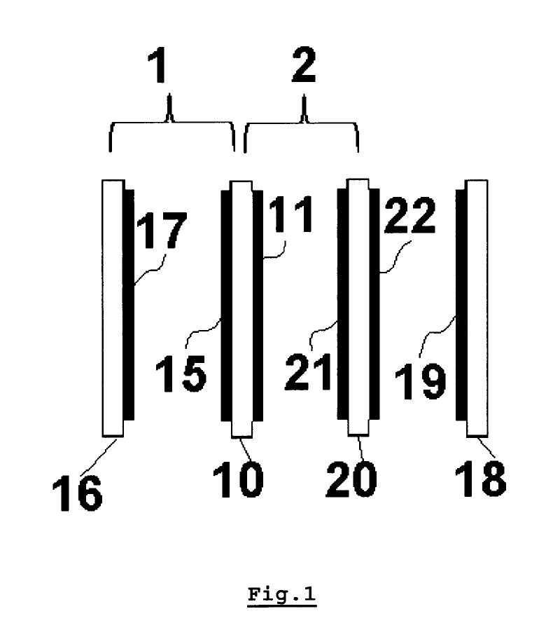

FIG. 1 illustrates the dosimetry device of the present invention comprising at least two ionisation chambers including at least two flexible films supporting one or more electrodes and called <<support films>> 10, 20 made in material of low density with a mean atomic weight of less than 20, having good flexibility and good resistance to radiation, such as biaxially-oriented polyethylene terephthalate better known as mylar, or poly(4,4′-oxydiphenylene-pyromellitimide better known as kapton, these materials not in any way forming a limitation to the present invention. Preferably, the at least two support films have a thickness of between one micrometre et one millimetre, more preferably between one micrometre and one hundred micrometres, further preferably between one micrometre and twenty micrometres.

At least two support films 10, 20 forming a first ionisation chamber are coated on their two surfaces with a layer of conductive material acting as electrode. Preferably, the said conductive material is deposited on the support film by a depositing technique so as to obtain a layer of conductive material of between one nanometre and one micron, preferably between 100 nanometres and one micron, more preferably between 100 and 500 nanometres. Preferably the said conductive material is a metal or graphite, more preferably a metal.

Compared with known support plates in the state of the art and generally obtained using the PCB technique, the support films of the present invention have the advantage that they produce less scattering and deterioration of the properties of the beam. Nonetheless the reduction in the thickness of the support films compared with those commonly used in the state of the art results in the onset of new problems, one first problem being the locating of the trace returning the signal to a signal measuring device, a second problem being a major capacitive effect at the films, and a third problem being the vibration of the films when they are subjected to an electric potential.

Conventionally, a collecting electrode is connected to a trace by a via passing through an insulating layer arranged between the surface of the electrode and the support plate, the said trace returning the signal to measuring equipment. For a support film whose thickness it is desired to minimise, this arrangement is not desirable.

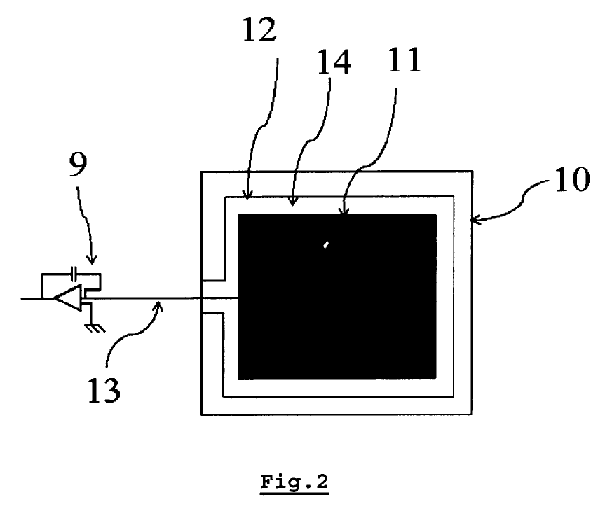

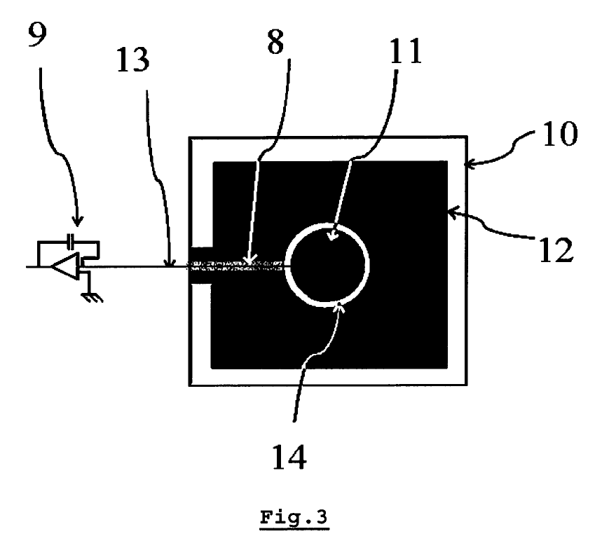

FIG. 2 illustrates a support film of the present invention comprising a collecting electrode 11 intended to measure a beam delivered using a dynamic technique, this type of electrode being called <<integral collecting electrode>>, the said collecting electrode 11 being connected to measuring electronics 9 by a trace 13 located on the same side of the support film as the electrode 11. The said trace is deposited on each support film using the same deposition technique as the one used for depositing the electrodes. Preferably, each collecting electrode and the trace connecting it to the measuring apparatus is separated from a guard layer 12 by a vacuum 14 or insulating resin 14 surrounding the perimeter of the collecting electrode. FIG. 3 shows a support film comprising a disc-shaped electrode intended for the measurement of a beam delivered by a passive technique. Since the trace of this collecting electrode must not be exposed to the beam otherwise it would provide measurement dependent on the field of this beam, this said trace is coated with a thin layer of insulating resin, itself coated with a thin layer of conductive material extending over the guard layer.

The capacitance of a capacitor is directly proportional to the area of the capacitor and inversely proportional to the distance separating the plates of the capacitor. A support film comprising a collecting electrode on one surface and a polarisation electrode on its other surface can be likened to a capacitor. For a support film having a thickness as in the device of the present invention, with a potential difference between the two electrodes located on the two sides of the film, the risk of breakdown of the film is very high. The breakdown of a film is a discharge occurring between the two insulated plates of the capacitor when too many charges have accumulated on one side of the capacitor, the discharge damaging the insulating layer of the capacitor.

Also, a major capacitive effect at the support film will result in delaying the transmission of charges towards the measuring electronics and increasing the detector response time. There is therefore a risk that detection of the dose deposited by the beam will be initiated at the time when the necessary dose has already been sent to the patient, and that an excess dose is sent damaging healthy tissues.

In the device shown in FIG. 1, the arrangement of the electrodes on the support films solves these capacitance problems. Each support film 10, 20 on its two surfaces comprises an electrode having the same polarisation. A first support film 10 comprises on its two surfaces a collecting electrode 11, 15 whose polarisation is preferably close to earth. The two surfaces of a second support film 20 each comprise a polarisation electrode 21, 22 preferably connected by a trace to a generator placed at a positive or negative potential. Each conductive trace connecting a polarisation electrode to the generator is located on the said side of the support film as the said polarisation electrode. In this manner two support films 10, 20 are obtained in which the two surfaces of one same support film are similarly polarised, which allows the capacitive effect to be greatly reduced either side of a support film.

Each support film 10, 20 is held in a support e.g. a support in epoxy resin, the said support guaranteeing good mechanical tensioning and good insulation of each support film. The two support films are secured so that a gap is created therebetween. The support comprises spacers for example having high electrical resistance, whose dimensions are calibrated with very small tolerances. The gaps separating the support films must have high guaranteed precision since the field, and hence the electrostatic force, depend on the applied electric voltage and on the distance between each support film.

Advantageously, the producing of a detector comprising flexible support films of relatively narrow thickness must also take into account the microphonic effect. The difference in potential created between two support films as thin as those of the present invention has the effect of buckling and/or vibrating these support films, which deteriorates the detection of the charges created by ionisation of the gas contained between the two support films through which a beam passes, since the gap between these two support films varies continuously. Similarly, external noise also produces a microphonic effect on said ionisation chamber; the device must therefore also minimise the contribution made by external noise.

To reduce this microphonic effect and more especially to obtain a uniform response of the collecting electrode over its entire surface, two plates or films 16, 18 are positioned either side of the ionisation chamber 1 formed by the two support films 10, 20. These two plates or films 16, 18 comprise electrodes 17, 19 placed at a potential chosen so as to set up an electrostatic force F_{E2} equilibrating with the electrostatic force F_{E1} created by polarisation of the support films 10, 20 of the ionisation chamber 1.

A first plate 16, preferably rigid, is positioned facing and parallel with the collecting electrode 15 located towards the outside of the ionisation chamber 1. This plate 16 comprises an electrode 17 which is placed at a potential chosen so as to equilibrate the electrostatic force F_{E1} applied to the support film 10 and resulting from the electric field set up by the difference in polarity between the collecting electrode 11 and the polarisation electrode 21 located towards the inside of the ionisation chamber 1. Preferably, the gap separating the electrode 17 contained on the first plate 16 from the electrode 15 contained on the support film 10, is identical to the gap separating the collecting 11 and polarisation 21 electrodes contained inside the ionisation chamber 1. More preferably, the voltage applied to the electrode 17 of plate 16 is equal to the voltage applied to the polarisation electrodes 21, 22 of the support film 20.

A second plate 18, which may or may not be rigid, is positioned facing and parallel with the support film 20 comprising the polarisation electrodes 21, 22. This second plate 18 comprises an electrode 19 placed at a potential chosen to equilibrate the electric force F_{E1} created by polarisation of the electrodes 21, 22 of the support plate 20. It is not necessary for this second plate 18 to be rigid if the electrode 19 contained on this plate 18 is not a collecting electrode, this electrode 19 together with electrode 22 therefore not forming an ionisation chamber.

Since the support film 10 comprises a collecting electrode 11, 15 on its two surfaces, charges created by ionisation of the gas by the beam are collected on the two sides of this film. Differences in the charges on each plate of one same film may lead to a slight capacitive effect, possibly interfering with measurement time at the measurement electronics. To avoid this inconvenience, the electric signal produced at the two collecting electrodes 11, 15 and resulting from ionisation of the gas is preferably physically summed before being sent to the measurement electronics. The support film 10 comprising the two collecting electrodes 11, 15 located on each side of this same film is therefore common to two ionisation chambers, a first ionisation chamber 1 being formed by the two support films 10, 20 and a second ionisation chamber 2 being formed by the support film 10 comprising the collecting electrodes and the rigid plate 16. It is therefore preferable in this case that these said ionisation chambers 1, 2 should have the same gap. This is why the plate 16 located facing the collecting electrode 15 of the support film 10 is a rigid plate, thereby reducing microphonic effects and guaranteeing a constant gap in the two ionisation chambers 1, 2 required for exact, precise dose measurement.

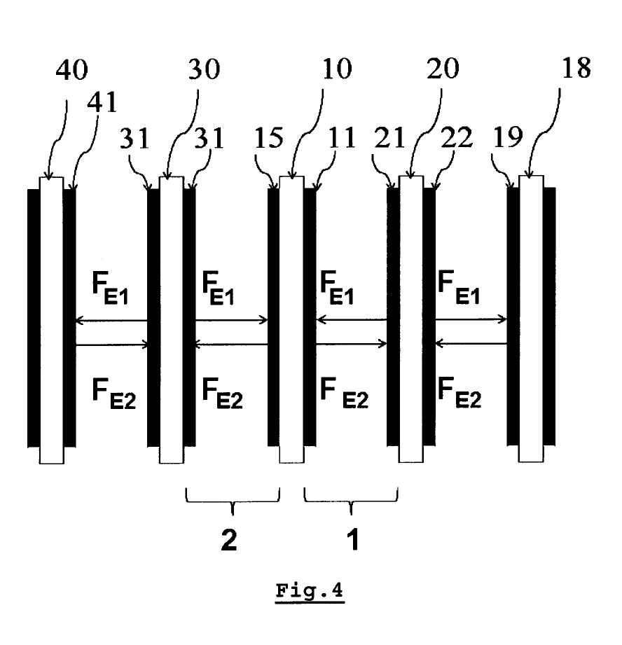

FIG. 4 shows one embodiment of the invention in which the rigid plate 16 has been replaced by a support film 30 having a polarisation electrode on its two surfaces, this support film preferably being identical to the support film 20 comprising a polarisation electrode on its two surfaces. This gives an assembly of two ionisation chambers 1, 2 comprising a collecting electrode common to these two ionisation chambers and collecting the same quantity of charges. Two films 18, 40 respectively comprise electrodes 19 and 41 preferably placed at identical potential or close to the potential of the collecting electrodes. These films 18, 40 are positioned either side of the said assembly of ionisation chambers and their electrodes create an equilibrating electrostatic force F_{E2} of opposite direction to the electrostatic forces F_{E1} applied to the support films 10, 30 comprising the polarisation electrodes placed at a negative potential for example. The films 18, 40 located either side of the said assembly of ionisation chambers 1, 2 must not necessarily be rigid since no charge is collected in the space formed by these films 18, 40 and the opposite-facing support films 20, 30.

As in the preceding case, the signals collected on the collecting electrode of the ionisation chamber 1 and 2 are summed and sent towards measurement electronics e.g. a charge integrator.

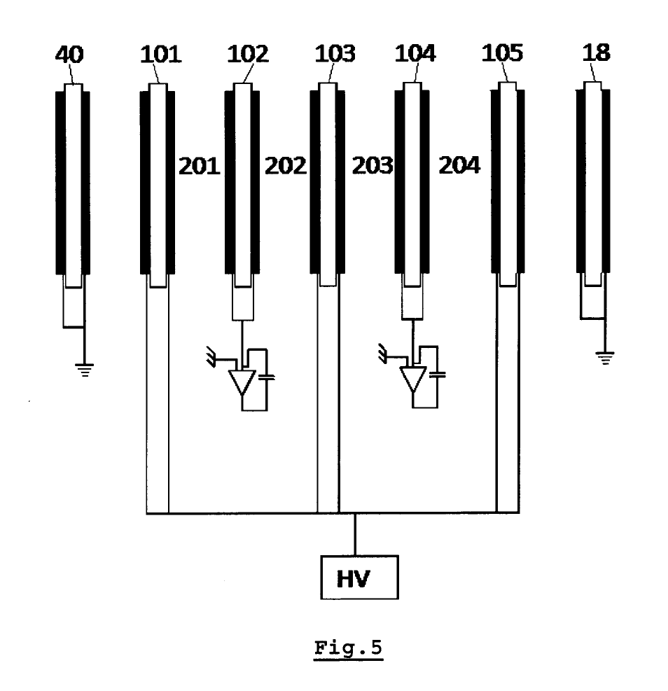

FIG. 5 illustrates another embodiment of the present invention dedicated to the so-called Pencil Beam Scanning technique. The device comprises an assembly of parallel ionisation chambers, each ionisation chamber comprising a flexible, thin support film on which a thin layer of conductive material is deposited by evaporation process which acts as collecting or polarisation electrode. Two support films 40, 18 on which electrodes are deposited by evaporation deposition are preferably earthed and positioned parallel either side of the said assembly of ionisation chambers. The assembly of ionisation chambers comprises two sub-assemblies of ionisation chambers. A first sub-assembly of ionisation chambers comprises two integral ionisation chambers 203, 204 measuring the dose deposited by the beam. This first sub-assembly of ionisation chambers comprises:
a first support film 105 comprising a polarisation electrode on its two surfaces;
a second support film 104 comprising a collecting electrode on its two surfaces, this support film being common to the two ionisation chambers 203, 204 of the first sub-assembly of ionisation chambers, the collecting electrode covering at least 90% of the support film, being surrounded by a guard electrode and whose structure is the one illustrated in FIG. 2;
a third support film 103 comprising a polarisation electrode on its two surfaces, this support film being common with the ionisation chamber 203 of the first sub-assembly of ionisation chambers and with one of the ionisation chambers 202 of the second sub-assembly of ionisation chambers.

The said collecting and polarisation electrodes extend over a region covering at least 90% of their support film so as to create and collect a maximum quantity of charges. A second sub-assembly of two ionisation chambers 201, 202 comprises:
the said support film 103;
a second support film 102 on which collecting electrodes are deposited in the form of strips, surrounded by a guard layer separated from these electrodes by an insulating material, so as to measure the beam field, each strip of one surface of the support film being connected to measurement electronics by a conductive trace located on the same side of the said second support film;
a third support film 101 comprising a polarisation electrode on its two surfaces.

The first sub-assembly of ionisation chambers 203, 204 lies adjacent the second sub-assembly of ionisation chambers 201, 202, one ionisation chamber 203 of the first sub-assembly having a support film 103 in common with an ionisation chamber 202 of the second sub-assembly of ionisation chambers. The first sub-assembly of ionisation chambers comprises two integral ionisation chambers 203, 204 formed by a support film 103, 105 comprising a polarisation electrode on surface side, and a support film 104 common with the two ionisation chambers 203, 204, the support film 104 comprising a collecting electrode on each surface.

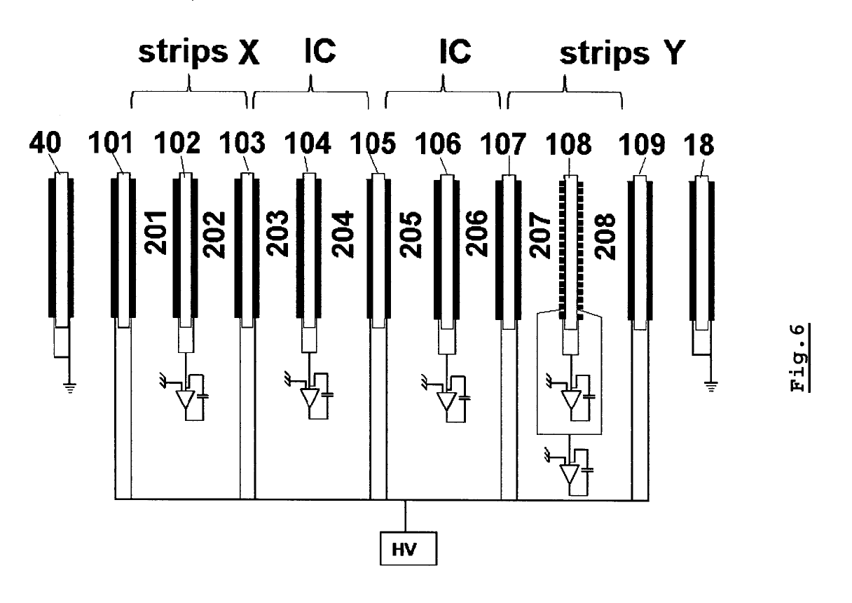

Preferably, the assembly of ionisation chambers of the device of the present invention comprises a third and a fourth sub-assembly of ionisation chambers as illustrated in FIG. 6. Preferably, the integral ionisation chambers 203, 204, 205, 206 are located towards the inside of the device whereas the ionisation chambers 201, 202, 207, 208 comprising electrodes in the form of strips are located towards the ends of the device. With this arrangement it is possible to have a stable precise signal in the integral ionisation chambers 203, 204, 205, 206 measuring the dose deposited by the beam. Preferably, a support film whether or not comprising a collecting electrode and earthed on each side is alternated with a support film comprising a polarisation electrode on each side. This redundancy of ionisation chambers allows repeat of measurements and ensures that the device functions correctly thereby guaranteeing maximum secure measuring of the dose delivered to the patient. In the event of breakdown of one of the support films, it is always possible to control the dose sent to the patient.

FIG. 6 shows two sub-assemblies of two adjacent, integral ionisation chambers 203, 204, 205, 206 in which:
a support film 104 is common to two ionisation chambers 203, 204 and on its two surfaces it comprises a collecting electrode;
a support film 105 is common to two ionisation chambers 204, 205 and each of its two surfaces comprises a polarisation electrode;
a support film 106 is common to two ionisation chambers 205, 206 and each of its two surfaces comprises a collecting electrode.

One sub-assembly of two ionisation chambers 201, 202 having in common a support film 102 comprising collecting electrodes in strip form on each of its two surfaces. One ionisation chamber 202 of this sub-assembly is positioned adjacent an integral ionisation chamber 203 and has in common with this ionisation chamber 202 a support film 103 comprising a polarisation electrode on each of its two surfaces.

A second sub-assembly of two ionisation chambers 207, 208 has in common a support film 108 comprising collecting electrodes in strip form on each of its two surfaces. For reasons of clarity, only two measurement electronic devices connected to the electrodes are illustrated. One ionisation chamber 207 of this sub-assembly is positioned adjacent an integral ionisation chamber 206 and has in common with this ionisation chamber 206 a support film 107 comprising a polarisation electrode on each of its two surfaces. Finally, one support film 18, 40 comprising an electrode facing the polarisation electrodes positioned towards the outside of the ionisation chambers 201, 208 that are located at the ends of the assembly of ionisation chambers allows the equilibrating of electrical forces due to polarisation of the electrodes 101, 103, 105, 107, 109 and contributes towards stabilising the support films of each ionisation chamber of the assembly.

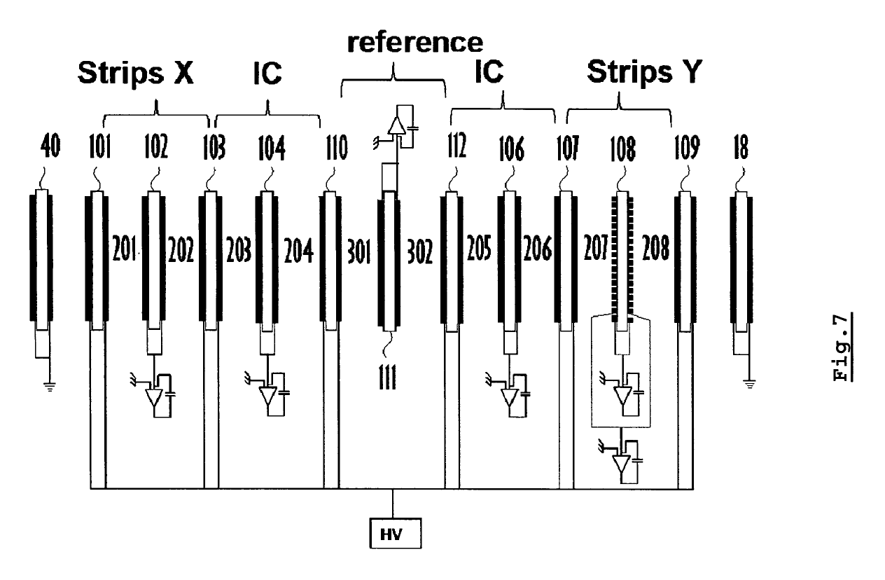

An additional sub-assembly of two ionisation chambers 301, 302 can be inserted in the said assembly of ionisation chambers as illustrated in FIG. 7. Preferably, this sub-assembly of ionisation chambers 301, 302 is arranged in the middle of the device, between the two sub-assemblies of integral ionisation chambers 203, 204 and 205, 206. This additional sub-assembly of ionisation chambers 301, 302 comprises a support film on which an electrode is deposited on the two sides of its surface, these electrodes equilibrating the electrostatic fields inside the device and able to be used as collecting electrodes to provide a reference signal when measuring, in a water phantom, a non-scanned beam for which it is desired to intercept the entirety of the flow of particles at the time of measurement in the said phantom. For conventional measurement in a water phantom it is difficult to position a reference chamber in a flow of particles without perturbing the measurement thereof. With one or more reference chambers in the device, said measurement is no longer perturbed.

Preferably the first sub-assembly of ionisation chambers 201, 202, through which the beam passes and positioned at the input of the device, comprises collecting electrodes in strip form oriented along an axis x orthogonal to the axis of the beam. The last sub-assembly of ionisation chambers 207, 208, through which the beam passes, comprises collecting electrodes in strip form oriented along an axis y orthogonal to the axis of the beam and to the said axis x.

This device can be placed at the output of a radiation unit and scarcely perturbs beam properties on account of its low water equivalent thickness, minimising the effects of angular and longitudinal scattering. It is possible for example to calculate the water equivalent thickness of a detector of the present invention by considering the last example of FIG. 6 which comprises 13 support films made of biaxially-oriented polyethylene terephthalate (mylar) e.g. 2.5 μm thick and coated on the two sides with a thin layer of gold or aluminium of thickness 200 nm for example, each support film being separated from the other by an air gap of 5 mm for example. The different parameters of this present example are reproduced in Table 1 for a beam of 200 MeV passing through this example of the device.

TABLE 1

| 1_{mylar} (cm) | ρ_{mylar} (g/cm³) | ${( \frac{1 \cdot d E}{ρ dx} )}_{mylar} ( MeV * {cm}^{2} / g )$ | WET_{mylar} (cm) |
|---|---|---|---|
| 2.5E−04 | 1.397 | 4.22E−03 | 2.25E−04 |
| 1_{gold} (cm) | ρ_{gold} (g/cm³) | ${( \frac{1 \cdot d E}{ρ dx} )}_{gold} ( MeV * {cm}^{2} / g )$ | WET_{gold} ( cm) |
| 2E−05 | 19 | 2.32E−03 | 1.94E−04 |
| 1ₐᵢᵣ (cm) | ρₐᵢᵣ | ${( \frac{1 \cdot d E}{ρ dx} )}_{air} ( MeV * {cm}^{2} / g )$ | WETₐᵢᵣ (cm) |
| 0.5 | 1.21E−03 | 3.95E−03 | 5.20E−04 |

This example of embodiment of the invention comprises 13 mylar films, 26 layers of gold and 12 air gaps. The water equivalent thickness of said detector is therefore (13*2, 25E-04)+(26*1, 94E-04)+(12*5, 20E-04)=0.014 cm for a detector length of about 6.13 cm. The thicknesses of the different materials are given solely as examples, other thicknesses and other materials possibly being chosen to implement the present invention. Similarly, some support films may differ from each other in respect of thickness and the materials chosen.

A device allowing measurement of the field and dose of a beam obtained using a so-called passive delivery technique can be obtained by reproducing the same structure as one of the devices described in the preceding embodiments, and by replacing the integral ionisation chambers whose collecting electrodes cover almost all the surface of the support films, by ionisation chambers whose collecting electrodes contained on the support films are disc-shaped.

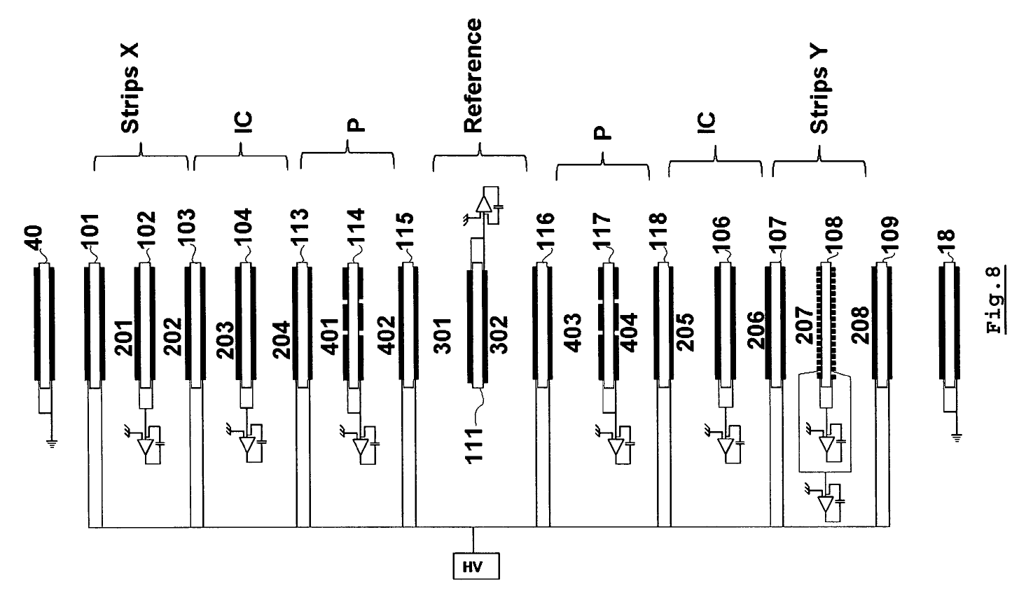

FIG. 8 illustrates another embodiment of the present invention allowing both dosimetry of beams of particles obtained using dynamic techniques and dosimetry of beams obtained using passive techniques. This embodiment illustrated in FIG. 8 comprises both integral ionisation chambers 203, 204, 205, 206 and ionisation chambers 401, 402, 403, 404 whose collecting electrodes are disc-shaped. In this embodiment, two sub-assemblies of two integral ionisation chambers and two sub-assemblies of tow ionisation chambers with disc-shaped collecting electrodes are arranged towards the middle of the device, for example symmetrically relative to an assembly of two reference ionisation chambers 301, 302. Said device may comprise an assembly of fourteen ionisation chambers also counting ionisation chambers 201, 202, 207, 208 which comprise electrodes in the form of strips. The device also comprises two support films 18, 40 positioned either side of this assembly of ionisation chambers and allowing equilibration of electrostatic forces and stabilization of the distances between each support film.

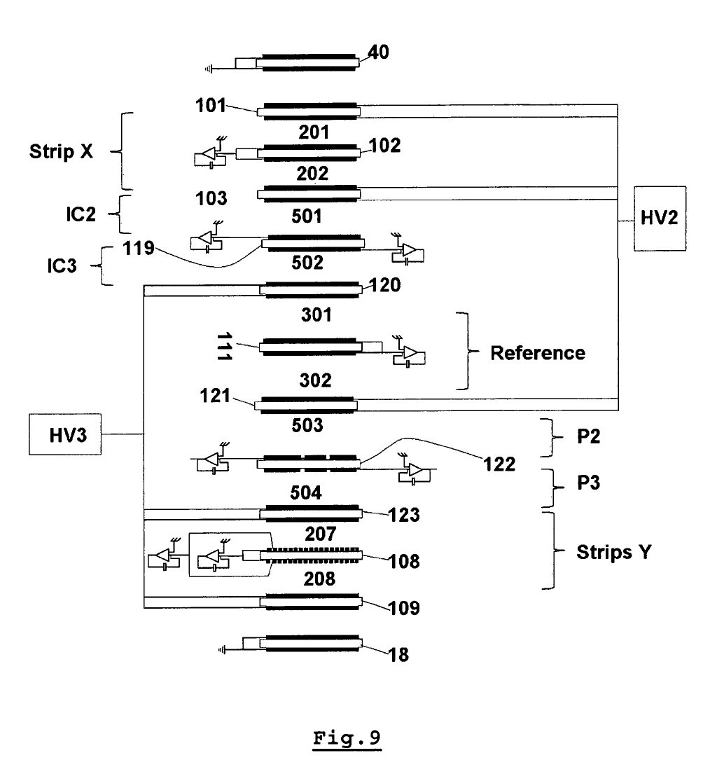

To reduce the number of ionisation chambers and support plates, whilst maintaining the redundancy characteristics of the device and the possibility of measuring beams obtained both with dynamic and passive delivery methods, each collecting electrode contained on a support film of an integral ionisation chamber and of an ionisation chamber with electrode of reduced size is connected to its own measurement electronics. One embodiment of the present invention is illustrated in FIG. 9 and comprises:
two first ionisation chambers 201, 202 comprising collecting electrodes in strip form, these ionisation chambers being formed by:
   a first support film 101 comprising a polarisation electrode on its two surfaces, each electrode being connected to a voltage generator HV2;
   a second support film 102 positioned facing the first support film 101 and comprising collecting electrodes in strip form on its two surfaces, arranged in identical manner on the two surfaces, each strip of one surface and each strip on the other side of the surface of the support film being connected to one same measurement electronics;
   a third support film 103 positioned facing the second support film 102 and comprising a polarisation electrode on its two surfaces, each electrode being connected to a voltage generator HV2;
A third ionisation chamber 501 formed by:
   the said third support film 103 and;
   a fourth support film 119 positioned facing the third support film 103 and comprising on the side facing the support film 103 an integral collecting electrode connected to its own measurement electronics;
A fourth ionisation chamber 502 formed by:
   a fifth support film 120 positioned facing the fourth support film 119 and comprising on its two surfaces a polarisation electrode connected to a voltage generator HV3;
   the fourth support film 119 comprising on the side facing the fifth support film 120 an integral collecting electrode connected to its own measurement electronics;
A fifth and a sixth reference ionisation chamber 301, 302 formed by:
   the said fifth support film 120;
   a sixth support film 111 positioned facing the fifth support film 120 and comprising a collecting electrode on its two surfaces;
   a seventh support film 121 positioned facing the sixth support film 111 and comprising on its two surfaces a polarisation electrode connected to a high voltage generator HV2;
A seventh ionisation chamber 503, formed by:
   the said seventh support film 121;
   an eighth support film 122 positioned facing the seventh support film 121 and comprising a disc-shaped collecting electrode surrounded by a guard, the electrode being connected to its own measurement electronics by a trace coated with an insulating resin, the electrode facing the said seventh support film;
An eighth ionisation chamber 504 formed by:
   a ninth support film 123 positioned facing the eighth support film 122 an comprising a polarisation electrode on its two surfaces;
   the said eighth support film 122 comprising a disc-shaped collecting electrode, surrounded by a guard, the electrode being connected its own measurement electronics by a trace coated with an insulating resin, the electrode facing the said ninth support film;
A ninth and a tenth ionisation chamber 207, 208 comprising electrodes in strip form, these ionisation chambers being formed by:
   the said ninth support film 123 comprising a polarisation electrode on its two surfaces, each electrode being connected to a voltage generator HV3;
   a tenth support film 108 positioned facing the ninth support film 123 and comprising on its two surfaces collecting electrodes in strip from arranged in identical manner on the two surfaces, each strip of one surface of the support film and its opposite facing strip on the other side of the surface of the support film being connected to one same measurement electronics;
   an eleventh support film 109 positioned facing the tenth support film 108 and comprising a polarisation electrode on its two surfaces, each electrode being connected to a voltage generator HV3.
      The assembly of these ionisation chambers is contained between two support films 40, 18 each comprising an electrode previously placed at the same potential as the collecting electrodes and positioned facing the support films of the first and tenth ionisation chamber.

This embodiment therefore overall comprises thirteen support plates and have a water equivalent thickness of 0.014 cm for device measuring about 6 cm and able to be used for measuring the dose and field of different types of beam. Although a single high voltage generator is sufficient to polarise all the polarisation electrodes, this embodiment of the present invention comprises two high voltage generators HV2, HV3 connected to the polarisation electrodes in the manner described above, in order to have redundancy of the ionisation chambers and to ensure measurement of the dose in the event of a problem with one of the two generators or in the event of breakdown of one of the support films comprising a polarisation electrode.

1. A device for the online monitoring of an ionising beam generated by a radiation source and delivered onto a target, the device comprising a plurality of support films arranged in parallel and separated from each other by a gap; the support films being positioned perpendicularly relative to the central axis of the ionising beam and forming a succession of ionisation chambers of which at least one ionisation chamber is formed using support films having a thickness equal to or less than 100 μm; each of the support films having on its two surfaces one or more electrodes set at a potential such that the two surfaces of each of the support films have the same polarity; the support films being arranged such that the successive support films have alternating polarisation; the device further having an additional component configured to equilibrate the electrostatic forces present inside the ionisation chamber formed using support films having a thickness equal to or less than 100 μm.

2. The device according to claim 1, wherein the at least one ionisation chamber is formed using support films having a thickness of less than 20 μm.

3. The device according to claim 1, wherein the additional component configured to equilibrate the electrostatic forces comprises a rigid plate, parallel to and facing the support film comprising a collecting electrode on each of its surfaces, and taking part in the formation of the ionisation chamber formed using support films having a thickness equal to or less than 100 μm; the rigid plate further comprising at least one electrode set at a potential capable of equilibrating the electrostatic forces present inside the ionisation chamber.

4. The device according to claim 1, wherein the additional component configured to equilibrate the electrostatic forces comprises a rigid or flexible plate parallel to and facing the support film comprising a polarisation electrode on each of its surfaces, and taking part in the formation of the ionisation chamber formed using support films having a thickness equal to or less than 100 μm; the rigid or flexible plate further comprising at least one electrode set at a potential capable of equilibrating the electrostatic forces present inside the ionisation chamber.

5. The device according to claim 1, wherein the gaps between each support film are constant.

6. The device according to claim 1, wherein at least one of the support films having a thickness equal to or less than 100 μm comprises an electrode on at least one of its surfaces.

7. The device according to claim 1 comprising support films having collecting electrodes on their two surfaces alternating with support films having polarisation electrodes on their two surfaces.

8. The device according to claim 7, wherein each collecting electrode is connected to measurement electronics by a trace located on the same side of the support film as the side comprising the collecting electrode.

9. The device according to claim 1 wherein some collecting electrodes assume the shape of strips arranged in parallel.

10. A device for measuring ionising beams, the device comprising a support film having two surfaces and having a thickness equal to or less than 100 μm, the support film comprising an electrode on at least one the surfaces.

11. The device according to claim 9, wherein the electrode is disc-shaped whose perimeter is separated by a gap or insulating resin from a guard layer which extends over the remainder of the support film, and wherein the disc-shaped electrode is connected to measurement electronics by a trace located on the same side of the support film as the side comprising the disc-shaped electrode, the trace being coated with an insulating resin, and the said insulating resin coated with a thin layer of conductive material which extends over the guard layer.

12. A method for online monitoring of an ionising beam generated by a radiation source and delivered to a target, the method comprising:
providing a plurality of support films arranged in parallel and separated from each other by a gap; the support films being positioned perpendicularly relative to the central axis of the ionising beam and forming a succession of ionisation chambers of which at least one ionisation chamber is formed using support films having a thickness equal to or less than 100 μm; each of the support films having one or more electrodes on its two surfaces;
setting each of the support films at a potential such that the two surfaces of each of the support films have the same polarity;
arranging the support films such that the successive support films have alternating polarisation;
determining the electrostatic forces present inside the ionisation chamber formed using support films having a thickness equal to or less than 100 μm; and
c) equilibrating the electrostatic forces.

13. The method according to claim 12, wherein the at least one ionisation chamber is formed using support films having a thickness less than 20 μm.

14. The method according to claim 12, wherein at least one of the support films having a thickness equal to or less than 100 μm comprises an electrode at least on one of its surfaces.

15. The method according to claim 12, wherein equilibrating the electrostatic forces is performed by a rigid or flexible plate comprising at least one electrode set at a potential capable of equilibrating the electrostatic forces present inside the ionisation chamber.

16. The method according to claim 12, wherein the equilibrating step further comprises applying a suitable voltage to the support films.

17. A method for online monitoring beams of particles delivered using passive delivery techniques, the method comprising utilizing the device according to claim 1.

18. A method for online monitoring beams of particles delivered using dynamic delivery techniques, the method comprising utilizing the device according to claim 1.

19. The device according to claim 6, wherein the electrode is a collecting electrode connected to measurement electronics by a trace located on the same side of the support film as the side comprising the electrode.

20. The device according to claim 10, wherein the electrode is a collecting electrode connected to measurement electronics by a trace located on the same side of the support film as the side comprising the electrode.
